(19) United States
(12) Patent Application Publication
(10) Pub. No.: US 2024/0180988 A1
(43) Pub. Date: Jun. 6, 2024

(54) USE OF MANGOSTEEN FRUIT SHELL EXTRACT IN THE PREPARATION OF A MEDICAMENT FOR TREATING BEDSORES

(71) Applicant: Xantho Biotechnology Co., LTD, Taipei City (TW)

(72) Inventors: Dai-Hua TSAI, Taipei City (TW); Shih-Yin CHEN, New Taipei City (TW); I-Pin CHUANG, New Taipei City (TW); Ku-Cheng CHEN, New Taipei City (TW); Yen-Ju CHEN, New Taipei City (TW)

(73) Assignee: Xantho Biotechnology Co., LTD, Taipei City, TW (TW)

(21) Appl. No.: 18/553,575

(22) PCT Filed: Dec. 21, 2021
(86) PCT No.: PCT/CN2021/140135

§ 371 Date: Oct. 2, 2023

Related U.S. Application Data
(60) Provisional application No. 63/172,659, filed on Apr. 9, 2021.

(51) Int. Cl. A61K 36/38; A61K 31/352; A61P 17/02

(52) U.S. Cl. CPC A61K 36/38; A61K 31/352; A61P 17/02; A61K 2236/331; A61K 2236/333

(57) ABSTRACT

A method for treating bedsores is provided, including administering a pharmaceutical composition includes an effective amount of mangosteen fruit shell extract to a subject suffering from bedsores, wherein the mangosteen fruit shell is outer shell of the mangosteen fruit shell and/or inner shell of the mangosteen fruit shell, and wherein the pharmaceutical composition is an external preparation.

### CROSS REFERENCE

This application is a national stage application, filed under 35 U.S.C. § 371, of International Patent Application No. PCT/CN2021/140135 filed on Dec. 21, 2021, and claims the benefit of priority from U.S. Provisional Application Ser. No. 63/172,659 filed on Apr. 9, 2021, the contents of each of which is incorporated by reference herein in its entirety.

### FIELD OF THE INVENTION

The present invention relates to a use of Mangosteen fruit shell extract in the preparation of a medicament for treating bedsores.

### BACKGROUND OF THE INVENTION

Skin is the largest organ of the human body. There are many types of skin diseases. Skin diseases may be acute (lasting only a few minutes to several hours) or chronic conditions, which may affect individuals for days, months, years and even the entire life. Skin diseases may be conditions caused by fungal, bacterial, or viral sources, or may be non-infectious, immune responses, such as inflammatory reactions with or without allergens, or idiopathic. Therefore, the symptoms of the skin diseases may vary and range from mild itching, redness and swelling to severe pus and nociceptive pain, for examples damaging ulceration. Skin diseases may impose significant impact on the quality of an individual's life.

Bedsores, also known as pressure ulcers, bed ulcers or decubitus ulcers, are localized damage on the skin and underlying tissue that usually occur over a bony prominence such as heels, hips and tailbone, in which usually resulting from prolonged pressure.

The risk factors of bedsores are usually considered to be diabetes mellitus, physical impairments, cognitive impairment, neuroesthesia, or older age. Bedsores also cause some complications, such as cellulitis, bone and joint infections, cancer, and septicemia.

In clinical, the international evidence-based clinical practice guideline in 2019 gives recommendations on strategies to treat bedsores, including the use of bed rest, pressure redistributing support surfaces, nutritional support, repositioning, wound care and biophysical agents. However, this guideline lacks teaching appropriate treating agents for treatment of bedsores.

Mangosteen has been used in the field of breast cancer prevention and muscle-related diseases, it has also been developed as nutritional supplements and cosmetics in daily lives, as well as used in the treatment of acute hepatitis, liver fibrosis and cirrhosis prevention.

Matsumoto et al., have studied α-mangostin, β-mangostin, γ-mangostin, and methyl-β-mangostin purified from Mangosteen fruit shells and investigated the inhibitory effect of this compound at various stages of the cell cycle, showing that this compound has anti-cell proliferative effect and anti-tumor effect (Bioorg. Med. Chem. 2005, 13, 6064-6069).

### SUMMARY OF THE INVENTION

The present invention provides a method for treating psoriasis in a subject, comprising administering a pharmaceutical composition comprises an effective amount of mangosteen fruit shell extract.

In a preferred embodiment, the Mangosteen fruit shell is water extract of Mangosteen fruit shell and/or alcohol extract of Mangosteen fruit shell.

In another preferred embodiment, the Mangosteen fruit shell is the outer shell of the Mangosteen fruit shell.

### DETAILED DESCRIPTION OF THE INVENTION

The present invention provides a use of a composition in preparation of a pharmaceutical composition for treating skin disorders.

Specifically, the present invention provides a use of a composition in preparation of a medicament for treating bedsores, wherein the composition comprises an effective amount of extract of Mangosteen fruit shell. The medicament can also be used for topical treatment use or for precision treatment use.

The present invention provides a method for treating bedsores in a subject, comprising administering a pharmaceutical composition comprises an effective amount of mangosteen fruit shell extract.

Mangosteen fruit shell contains a softer inner shell and a harder outer shell.

In a preferred embodiment, the Mangosteen fruit shell is extracted with a solvent which is selected from the group consisting of methanol, ethanol, n-propanol, 2-propanol, n-butanol, acetone, ethyl acetate and water.

In another preferred embodiment, the extract of Mangosteen fruit shell is water extract of Mangosteen fruit shell and/or alcohol extract of Mangosteen fruit shell.

In a preferred embodiment, the extract of Mangosteen fruit shell is a Mangosteen fruit shell water extract.

In another preferred embodiment, the extract of Mangosteen fruit shell is Mangosteen fruit shell alcohol extract.

In a preferred embodiment, the Mangosteen fruit shell is the inner shell/outer shell of the Mangosteen fruit shell and/or the whole shell of the Mangosteen fruit shell.

In another preferred embodiment, the Mangosteen fruit shell is the outer shell of the Mangosteen fruit shell.

In a preferred embodiment, the compositions of the present invention can be oral or parenteral preparations, the parenteral preparations can be external preparations which can be creams, pastes, ointments, gels, wash lotions or patches.

In a preferred embodiment, the extract of Mangosteen fruit shell of the present invention comprises α-mangostin and γ-mangostin.

In another preferred embodiment, the water extract of Mangosteen fruit shell of the present invention comprises α-mangostin and γ-mangostin.

In yet another preferred embodiment, the alcohol extract of Mangosteen fruit shell of the present invention comprises α-mangostin and γ-mangostin.

As used herein, the term “Effective amount” is the amount that can achieve effective results when administered to an individual, or that has the desired activity in vivo or in vitro. In the present invention, the term “Effective amount” is calculated by the ratio of the weight of the extract to the total weight of the composition. In the case of bedsores, as compared to no treatment, effective clinical outcomes include amelioration of the extent or severity of the symptoms associated with the disease or condition, and/or prolonging the life of an individual and/or improvement of the quality of life of the individual. The exact amount of compound administered to an individual will depend on the type and severity of the disease or symptoms and on the individual characteristics such as the general health of the individual, age, sex, weight, and drug tolerance of the individual. It is also dictated by the conditions, severity and types of the inflammatory disorder, the autoimmune disorder and the allergic disorder, or the desired immunosuppressive effect. Those skilled in the art will be able to determine the appropriate dose based on these and other factors.

In an embodiment, the effective amount of extract of Mangosteen fruit shell is 0.5% (w/w) to 10% (w/w). In a preferred embodiment, the effective amount of extract of Mangosteen fruit shell is 1% (w/w) to 8% (w/w). In a most preferred embodiment, the effective amount of extract of Mangosteen fruit shell is 1.25% (w/w) to 5% (w/w).

The pharmaceutical composition of the present invention can be formulated into various forms of oral or parenteral preparations. Oral preparations can be formulated as solid preparations such as powders, granules, troches, capsules, etc., or formulated as liquid preparations such as suspensions, emulsions, syrups, etc. Parenteral preparations can be formulated as external preparations such as creams, ointments, gels, wash lotions, patches, etc., or as inhalants, aerosols, suppositories, etc.

The pharmaceutical composition of the present invention can comprise pharmaceutically acceptable excipients, especially can further comprise predetermined solvents or oils, pH adjuster and if desired, can further comprise a dispersant.

Examples of solvents used in the present invention include, but are not limited to, water, ethanol, isopropanol, 1,3-butanediol, propylene glycol, glycerin, etc.

Examples of oils used in the present invention are selected from the group consisting of, but are not limited to, corn oil, sesame oil, flaxseed oil, cottonseed oil, soybean oil, peanut oil, mono-glycerides, di-glycerides, tri-glycerides, mineral oil, squalene, jojoba oil, olive oil, evening primrose oil, borage oil, grape seed oil, coconut oil, sunflower oil, shea butter, and any combinations thereof.

Solvents and oils can be used alone or in any combinations thereof.

Examples of useful dispersants include, but are not limited to, lecithin, organic monoglycerides, sorbitan fatty acid esters, polyoxyethylene fatty acid esters, sorbitan stearate, etc. These raw materials can also be used alone or in any combinations thereof.

If desired, the composition further comprises additional materials such as antimicrobials or preservatives.

In the meantime, it is known that an active ingredient can be used simultaneously with the composition as long as it does not have any adverse effects on the pharmaceutical activity of the composition of the present invention. For example, ceramide moisturizers are commonly used as conventional agents for atopic dermatitis, or liquid ingredients such as hydrocortisone steroids, vitamin A derivatives such as vitamin A palmitate and/or tocopherol, etc., can be used with the composition.

When the pharmaceutical composition is used as an external preparation, an appropriate external skin preparation can be used as a base material, and an aqueous solution, a non-aqueous solvent, a suspension, an emulsion or a lyophilized preparation, etc., can be used and sterilized according to known methods.

In practical use of the provided or administered composition of the present invention, the effective amount can be determined depending on various factors such as the route of administration, the age, sex, and weight of the patient, the severity of the disease, and the type of medicament as the active ingredient.

In the case where the composition of the present invention can be a food or a cosmetic composition, the composition can be prepared by appropriate addition of at least one food supplement or a cosmetically acceptable carrier.

The food composition can be used in or added to, for example, healthy foods. As used herein, the term “healthy food” refers to a food product containing the composition of the present invention that has an enhanced function as compared to general food products. Healthy foods can be prepared by adding a general food to the composition or by encapsulation, pulverization or suspension liquefaction.

The cosmetic composition can be added alone or together with other cosmetic ingredients, or can be appropriately used according to other known methods. Cosmetics include, but are not limited to, aftershaves, lotions, creams, facial masks and color makeups.

Cosmetic compositions can be formulated into various forms of compositions, such as gels, creams, ointments, etc. The compositions in the form of gels, creams and ointments can be appropriately prepared according to the form of the composition by using known methods, and by addition of known softeners, emulsifiers and thickeners or other materials known in the art.

The gel-form composition can be prepared, for example, by addition of a softener such as trimethylolpropane, polyethylene glycol and glycerol, for example, a solvent of propylene glycol, ethanol and isocetyl alcohol, and pure water.

The preparation of the compositions in the form of creams can be carried out, for example, by addition of fatty alcohols such as stearyl alcohol, myristyl alcohol, behenyl alcohol, resveratrol, isostearyl alcohol and isocetyl alcohol; emulsifiers such as lipids, such as lecithin, phosphatidylcholine, phosphatidylethanolamine, phosphatidyl serine, phosphoinositide and derivatives thereof, glyceryl stearate, sorbitol palmitate, sorbitol stearate, etc; natural fats And oils such as avocado oil, almond oil, babassu oil, borage oil, camellia oil, etc; lipid compositions such as ceramides, cholesterol, fatty acids, phytosphingosine, lecithin, etc; solvents, such as propylene glycol, etc; and pure water.

The preparation of the compositions in the form of ointments can be carried out, for example, by addition of emollients, emulsifiers and waxes, for example microcrystalline wax, paraffin, ceresin, beeswax, spermaceti, petrolatum, etc.

In another aspect, the present invention provides a method for using the composition to prepare a medicament for treating or alleviating bedsores. As used herein, the term “treating or alleviating” means that when a patient uses a medicament, it stops or delays the course or symptoms of the disease.

### BRIEF DESCRIPTION OF THE DRAWINGS

The patent or application file contains at least one drawing executed in color. Copies of this patent or patent application publication with color drawing(s) will be provided by the Office upon request and payment of the necessary fee.

FIG. 1 illustrates the animal experiment flowchart of the invention. FIG. 1(A) shows the timeline of the animal experiment. FIG. 1(B) demonstrated the establishment of mouse bedsores model.

FIG. 2 illustrates the area of stage 3 bedsores after 7 days treatment. CTL: sham group; Placebo: vehicle group; Whole-shell extracts: Xantho sample groups with the effective amount of 0.625% (w/w), 1.25% (w/w), 2.5% (w/w), and 5% (w/w). Results are expressed as the mean±SEM. *P<0.05, **P<0.01, Student's t test.

FIG. 3 illustrates the image of stage 3 bedsores healing after 7 days treatment. CTL: sham group; Placebo: vehicle group; Whole-shell extracts: Xantho sample groups with the effective amount of 0.625% (w/w), 1.25% (w/w), 2.5% (w/w), and 5% (w/w).

FIG. 4 illustrates histological evaluations of the mice's skin showed significant changes in 2.5% (w/w) group compared with placebo. *P<0.05, **P<0.01, ***P<0.001, Student's t test. FIG. 4(A) The inflammation score; FIG. 4(B) The re-epithelialization score; FIG. 4(C) The Granulation of tissue score;

FIG. 4(D) The Angiogenesis score. CTL: sham group; Placebo: vehicle group; Mangosteen fruit shell extracts: Xantho sample groups.

FIG. 5 illustrates the area of stage 3 bedsores after 7 days treatment. CTL: sham group; Placebo: vehicle group; Mangosteen fruit shell extracts: Xantho sample groups with the effective amount of 2.5% (w/w), I: inner-shell extracts, O: outer-shell extracts, W: whole-shell extracts. Results are expressed as the mean±SEM. *P<0.05, Student's t test.

FIG. 6 illustrates the image of stage 3 bedsores healing after 7 days treatment. CTL: sham group; Placebo: vehicle group; Mangosteen fruit shell extracts: Xantho sample groups with the effective amount of 2.5% (w/w), I: inner-shell extracts, O: outer-shell extracts, W: whole-shell extracts.

### EMBODIMENTS

The examples below are non-limiting and are merely representative of various aspects and features of the present invention.

### EXAMPLES

### Preparation of Pharmaceutical Compositions

The Mangosteen fruit shell extract was prepared by following the preparation steps recited in U.S. Pat. No. 10,383,906B2.

Briefly, Mangosteen fruit shell was collected and dried to 50% to 95%, extracted with a solvent (such as water or 10% to 95% alcohol), and concentrated to obtain an extract of Mangosteen fruit shell.

The outer shell and inner shell of the Mangosteen fruit shell were separated, the outer shell of the Mangosteen fruit shell and the inner shell of the Mangosteen fruit shell were respectively dried to 50% to 95%, and extracted with a solvent (such as water or 10% to 95% alcohol), then concentrated to obtain an extract of mangosteen outer shell and an extract of mangosteen inner shell.

Different concentrations of pastes or ointments were prepared from the Mangosteen fruit shell alcohol and water extract, the mangosteen inner shell, outer shell alcohol and water extract. They are herein called “Xantho sample” in the invention.

### Animal Experiments

7-week-old BALB/c mice (purchased from LESCO Biotech), body weight 24 to 30 g, were used in the animals experiments and were quarantined by veterinarians for one week before entering the breeding room. The temperature of the breeding room was set at 21±2° C., the humidity was set at 30 to 70%, and the environment was set for 12/12 hours light-dark cycle with unlimited supply of food and water.

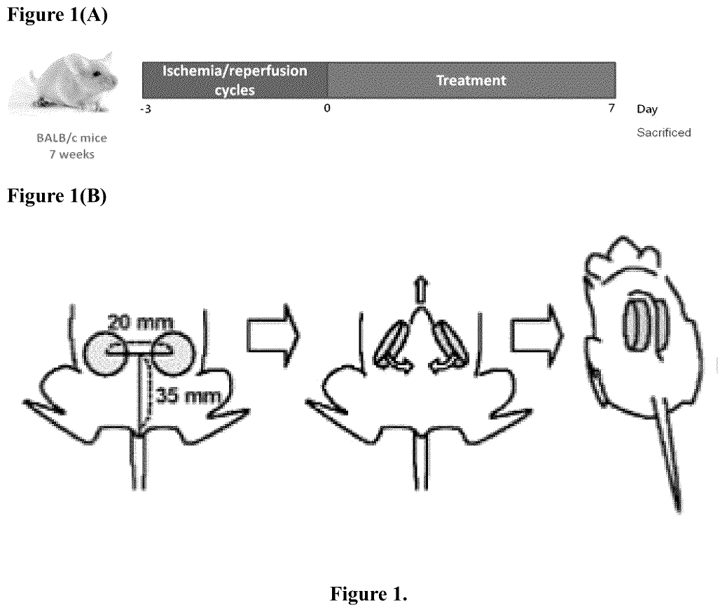

The animal experiment flowchart of the invention is shown in FIG. 1(A). A mouse bedsores model was developed according to the method described by Stadler et al., with a slight modification. The mice were sedated using isoflurane and O₂ for approximately 50-60 seconds. The dorsal hair was shaved. A template was used to mark the location of the magnetic plates to assure a consistent placement on each animal. The dorsal skin was sandwiched and compressed by the attraction between 2 round ceramic magnetic plates that had 12 mm diameter and were 5.0 mm thick, with 1000 G magnetic force (see FIG. 1(B)).

Three ischemia-reperfusion (IR) cycles were used in each animal to initiate bedsores formation. A single ischemia-reperfusion cycle consisted of a 6-h period of magnet placement followed by a release or rest period of 18 h. Animals were not immobilized, anesthetized, or otherwise treated during the IR cycles.

The animals were allowed food and water ad libitum. Totally 36 mice were randomly divided into 6 groups (6 mice in each group): a sham group (labeled CTL), a vehicle group treated with excipient (water and other emulsifiers; labeled placebo), and four Xantho sample groups treated with the test substance of Mangosteen fruit whole-shell extracts (labeled whole-shell extracts) with the effective amount of 0.625% (w/w), 1.25% (w/w), 2.5% (w/w), 5% (w/w) by applying to the wound. After stage 3 bedsores formation, the wounds were treated with each sample (40.25 mg/cm2) daily for 7 days. Several parameters, including: ulcer healing, body weights, food and water intakes, were observed on day 0, day 4, and day 7. The wound areas of each group were photographed and then were analyzed by using the ImageJ software (ImageJ Fiji, USA). The stage of the ulcer was graded using a standardized grading scale as shown in Table 1, which is widely used in clinical practice. Changes in skin color and skin integrity were graded on a scale of 0-4, where 0 is assigned to intact normal skin and 4 is assigned to lesions with full-thickness loss of skin and subcutaneous tissue and/or scar formation. The mean ulcer stage and standard deviation was calculated.

TABLE 1
Standardized pressure ulcer grading scale

| Ulcer stage | Description |
|---|---|
| 0 | Intact, normal, skin with normal capillary refill |
| 1 | Intact skin, nonblanchable erythema |
| 2 | Superficial/partial skin loss involving epidermis and dermis |
| 3 | Full-thickness loss with damage and |
| | necrosis of subcutaneous tissue |
| 4 | Full-thickness loss with extensive |
| | destruction, tissue necrosis, |
| | and with exposure of muscle and bone |

### Histopathology

Skin tissue from each mouse were fixed in 10% phosphate-buffered formalin, and then embedded in paraffin blocks. Sections of 5-mm thick of paraffin-embedded tissues were mounted on glass slides, rehydrated with distilled water, and stained with hematoxylin and eosin. As part of the histological evaluation, all slides were examined by a pathologist without knowledge of the previous treatment, using masked slides under the microscope at a 50-fold magnification (LEICA DM2700 M, USA). The parameters measured were inflammation, re-epithelialization, granulation of tissue, and angiogenesis. The histological score system is shown in Table 2.

TABLE 2

| | | | Granulation | |
|---|---|---|---|---|
| Score | Inflammation | Re-epithelialization | of tissue | Angiogenesis |
| 0, − | <10% | Absent | Inflammatory | Absence of |
| | | | tissue | angiogenesis, |
| | | | | inflammation, and |
| | | | | hemorrhage |
| 1, + | 11-25% | Little epidermal and | Thin granular | Altered angiogenesis |
| | | dermal organization | layer | (1-2 vessels per site) |
| | | | | characterized by a |
| | | | | high degree of edema, |
| | | | | hemorrhage, and |
| | | | | occasional congestion |
| | | | | and thrombosis |
| 2, ++ | 26-50% | Moderate epidermal | Moderate | Few newly formed |
| | | and dermal | granulation | capillary vessels (3-6 |
| | | organization | layer | per site), moderate |
| | | | | degree of edema and |
| | | | | hemorrhage. |
| | | | | Occasional congestion |
| | | | | and inter vascular |
| | | | | fibrin deposition; |
| | | | | absence of thrombosis |
| 3, +++ | 51-75% | Complete remodeling | Thick | Newly formed |
| | | of epidermis and | granulation | capillary vessels (7-10 |
| | | dermis | layer | per site), moderate |
| | | | | degree of perivascular |
| | | | | and interstitial edema |
| | | | | and congestion. |
| | | | | Absence of |
| | | | | thrombosis and |
| | | | | hemorrhage |

### Statistical Analysis

The results were presented as the mean±standard deviation. Statistical analysis was performed by one-way analysis of variance with Dunnett's post-hoc test using GraphPad Prism (version 8.0). P values of statistical significance are represented as *P<0.05, **P<0.01 and ***P<0.001.

### Result

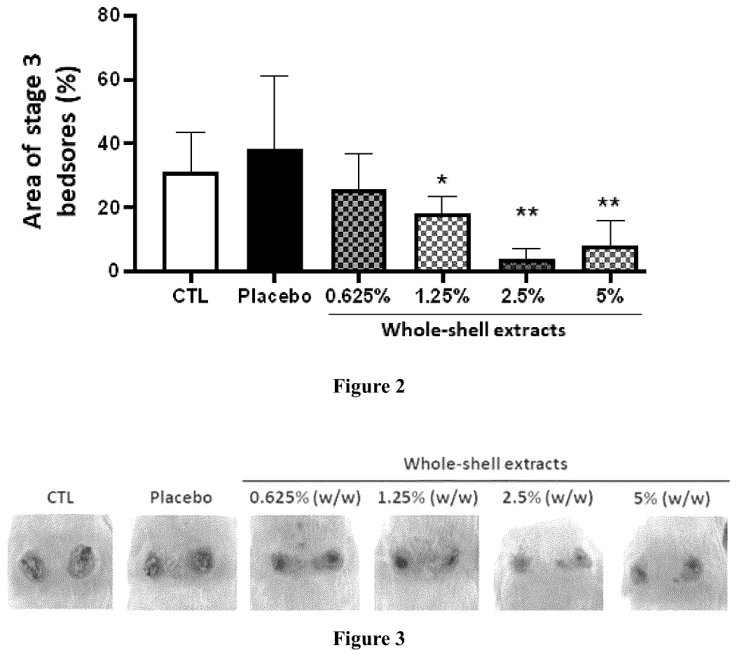

After 7 days treatment, administering whole-shell extracts with the effective amount of 1.25% (w/w), 2.5% (w/w), and 5% (w/w) resulted significant size decreasing of stage 3 ulcers (see FIG. 2 and FIG. 3). Particularly, the group given whole-shell extract with the effective amount of 2.5% (w/w) showed much better effect. Compared with CTL: *, p<0.05; **, p<0.01.

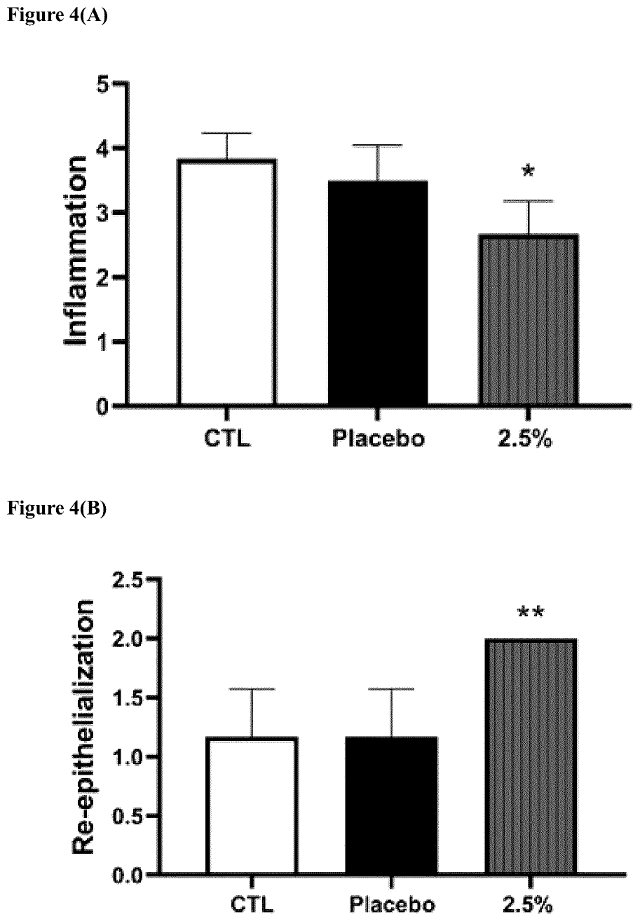

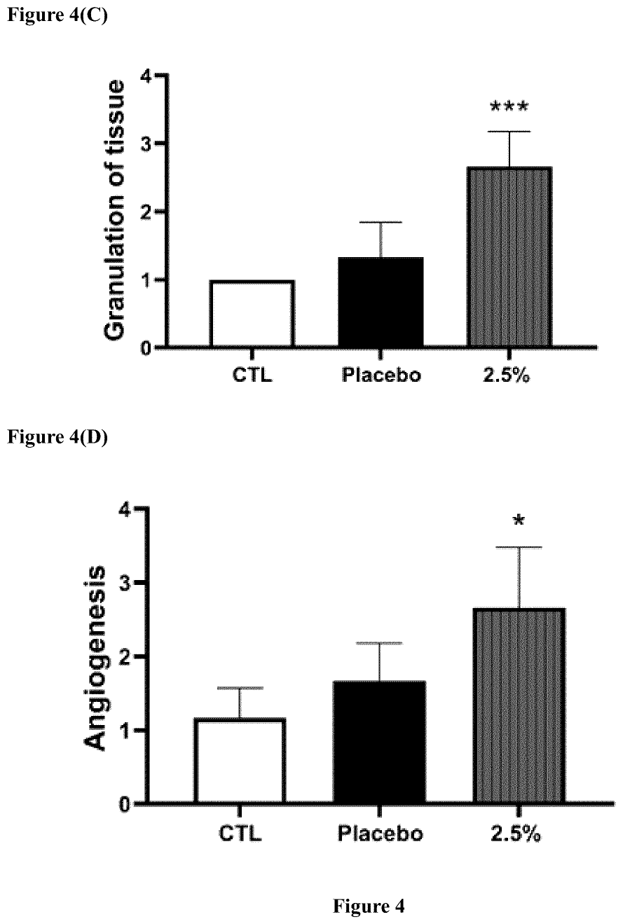

For the evaluation of the histological score of bedsores by treating with 2.5% whole-shell extracts compared to placebo group, the results were shown in FIG. 4. The inflammation score (FIG. 4A) showed that, except for 2.5% whole-shell extracts which dropped to 2.67, the rest of the group's score were between 3.83-4. The re-epithelialization score (FIG. 4B) showed, except for 2.5% whole-shell extracts which increased to 2, the rest of the group's score were between 1-1.17. The Granulation of tissue score (FIG. 4C) showed, except for 2.5% whole-shell extracts which increased to 2, the rest of the group's score were between 1-1.33. The Angiogenesis score (FIG. 4D) showed, except for 2.5% whole-shell extracts which increased to 2.67, the rest of the group's score were between 1-1.67. The results indicate that 2.5% whole-shell extracts the most effective in the healing of wounds and bedsores.

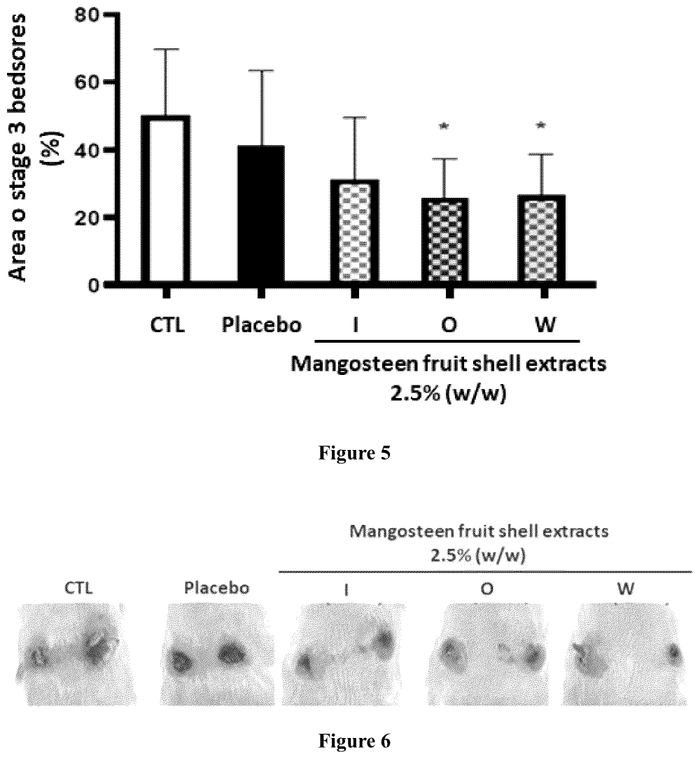

Further confirming the effect of 2.5% (w/w) mangosteen fruit shell extract, 30 7-week-old BALB/c mice were divided into 5 groups, a sham group (labeled CTL), a vehicle group (labeled placebo), and three Xantho sample groups (labeled Mangosteen fruit shell extracts; I: inner-shell extracts, O: outer-shell extracts, W: whole-shell extracts), 6 in each group. After stage 3 bedsores formation, the wounds were treated with each sample (40.25 mg/cm2) daily for 7 days. Several parameters, including: ulcer healing, body weights, food and water intakes, were observed on day 0, day 4, and day 7. The wound areas of each group were photographed and then were analyzed by using the ImageJ software (ImageJ Fiji, USA). As shown in FIG. 5 and FIG. 6, 2.5% (w/w) outer-shell extracts and whole-shell extracts decrease the size of stage 3 ulcers significantly, it seemed that the outer-shell extracts having the most potential treating effect to bedsores.

While the invention has been described and exemplified in sufficient detail for those skilled in this art to make and use it, various alternatives, modifications, and improvements should be apparent without departing from the spirit and scope of the invention.

One skilled in the art readily appreciates that the present invention is well adapted to carry out the objects and obtain the ends and advantages mentioned, as well as those inherent therein. The cells, animals, and processes and methods for producing them are representative of preferred embodiments, are exemplary, and are not intended as limitations on the scope of the invention. Modifications therein and other uses will occur to those skilled in the art. These modifications are encompassed within the spirit of the invention and are defined by the scope of the claims.

1. A method for treating bedsores, comprising administering a pharmaceutical composition comprises an effective amount of mangosteen fruit shell extract to a subject suffering from bedsores.

2. The method of claim 1, wherein the mangosteen fruit shell extract is water extract of mangosteen fruit shell and/or alcohol extract of mangosteen fruit shell.

3. The method of claim 1, wherein the mangosteen fruit shell is outer shell of the mangosteen fruit shell and/or inner shell of the mangosteen fruit shell.

4. The method of claim 1, wherein the mangosteen fruit shell is outer shell of the mangosteen fruit shell.

5. The method of claim 1, wherein the mangosteen fruit shell extract comprises α-mangostin and γ-mangostin.

6. The method of claim 1, wherein the composition is a parenteral preparation.

7. The method of claim 6, wherein the parenteral preparation is an external preparation.

8. The method of claim 1, wherein the effective amount of Mangosteen fruit shell extract is 0.5% w/w to 10% w/w.

9. The method of claim 1, wherein the effective amount of Mangosteen fruit shell extract is 1% w/w to 8% w/w.

10. The method of claim 1, wherein the effective amount of Mangosteen fruit shell extract is 1.25% w/w to 5% w/w.

11. (canceled)
